(19) United States
(12) Patent Application Publication
(10) Pub. No.: US 2022/0265221 A1
(43) Pub. Date: Aug. 25, 2022

(54) METHOD AND SYSTEM FOR MONITORING PHYSIOLOGICAL SIGNALS

(71) Applicant: MYBRAIN TECHNOLOGIES, Issy-les-Moulineaux (FR)

(72) Inventors: Xavier NAVARRO-SUNE, Paris (FR); Nicolas POURCHIER, Roquevaire (FR); Fabrice VAILHEN, Paris (FR); Yohan ATTAL, Paris (FR)

(73) Assignee: MYBRAIN TECHNOLOGIES, Issy-les-Moulineaux (FR)

(21) Appl. No.: 17/622,476

(22) PCT Filed: Sep. 17, 2020
(86) PCT No.: PCT/EP2020/075931

§ 371 Date: Dec. 23, 2021

(30) Foreign Application Priority Data
Sep. 20, 2019 (EP) 19306151.2

(51) Int. Cl. A61B 5/00; A61B 7/00; A61B 5/369; A61B 5/0205

(52) U.S. Cl. CPC A61B 5/7278; A61B 5/6803; A61B 7/003; A61B 5/726; A61B 5/7257; A61B 5/369; A61B 5/721; A61B 5/0205; A61B 2562/0219; A61B 2560/0247; A61B 2562/0204

(57) ABSTRACT

A method for monitoring physiological signals of a subject from sounds produced by the subject, including: receiving recorded sounds, including sounds from the subject's chest and being transmitted by the subject's biological tissues to the subject's ears, the recorded sounds being recorded by sound recording element(s) positioned inside earcup(s) of headphones worn by the subject; receiving signals from an accelerometer and a gyroscope being recorded simultaneously with the recorded sounds; detecting heart beats from the cardiac peaks sounds and calculating inter-beat intervals from the heart beats; extracting a first estimation of the breathing signal from the inter-beat intervals presenting respiratory sinus arrhythmia; extracting a second estimation of the breathing signal from residual sounds; extracting a third estimation of the breathing signal and motion artifacts from the signals of the accelerometer and the gyroscope; calculating the breathing signal by combining the first, second and third estimations of the breathing signal.

### FIELD OF INVENTION

The present invention relates to the field of acoustic signal analysis. In particular, the present invention relates to the field of the analysis acoustic signal produced by a subject so as to monitor at least one physiological signal of said subject.

### BACKGROUND OF INVENTION

Nowadays innovations in wearable sensors have opened the possibility for monitoring human physiological functions out of the clinic environment. Early attempts in this direction employed actigraphy, which uses inertial sensors to measure activity of various body parts, with applications in sports and general health. More recent efforts have focused on recording cardiac activity, either electrically, via electrocardiograms (ECG), or optically, through photoplethysmograms (PPG). These require either a chest-strap or wrist-band, with the latter considered inconspicuous and comfortable to wear for most people, and the former quite uncomfortable.

The development of wearable devices for other modalities, such as respiration and neural activity, has been largely hampered by the inconvenience of their respective form factors—a chest-strap within respiration monitors is uncomfortable for long-term use, while the electroencephalogram (EEG) recorded from the scalp is cumbersome to set up, stigmatizing when out-of-the-clinic, and prone to artifacts.

Thanks to the recent advances, the health monitoring solutions starts to consider more than one single sensing modality. For example, US patent application 2018/014741 describes a wearable physiological monitoring device including plural EEG electrodes, an optical sensor comprised in an ear-plug structure configured to be mounted inside an ear of a user. This device is configured to measure the electroencephalographic signals using the EEG electrodes and the cardiac signal from the optical sensor.

Actual health monitoring solutions either not suitable for out-of-the-clinic measurement or uncomfortable for long-term use.

It is these drawbacks that the invention is intended more particularly to remedy by proposing an apparatus and a method for monitoring physiological signals of a subject from sounds produced by the subject.

### SUMMARY

The present invention relates to a computer-implemented method for providing physiological signals of a subject, said method comprising:
receiving recorded sounds comprising sounds originating from a chest of the subject and being transmitted by biological tissues of the subject to the ears of the subject, wherein said recorded sounds previously recorded by at least one sound recording element positioned inside at least one earcup of headphones worn by the subject;
receiving signals from an accelerometer and a gyroscope which have been recorded simultaneously with the recorded sounds;
extracting from the recorded sounds cardiac peaks, corresponding to systolic and diastolic sounds, and residual sounds comprising information generated by the breathing signal;
detecting heart beats from the cardiac peaks sounds and calculating inter-beat intervals from the heart beats;
extracting a first estimation of the breathing signal from the inter-beat intervals presenting respiratory sinus arrhythmia;
extracting a second estimation of the breathing signal from residual sounds;
extracting a third estimation of the breathing signal and motion artifacts from the signals of the accelerometer and the gyroscope;
calculating the breathing signal by combining the first, the second and the third estimation of the breathing signal, and
providing the breathing signal and inter-beat intervals for health monitoring.

The present invention advantageously allows to obtain from sound recorded in proximity of the ear canal and the inertial motion signals a robust estimation of the cardiac and respiratory signal. If one side the cardiac signal is relatively intense to be easily detected from the recorded sounds, the evaluation of respiratory signals is particularly challenging due to their low intensity. The present method thanks to the fusion of three different estimations of the breathing signal is advantageously able to provide a reliable measure of the breathing signal.

The fact that the accelerometer and a gyroscope signals are recorded simultaneously to the recorded sounds advantageously allows to combine the third estimation of the breathing signal (obtained by the accelerometer and a gyroscope signal) to the first and second estimations resulting from the analysis of the recorded sounds.

According to one embodiment, the method further comprises, after receiving recorded sounds, receiving as well sounds propagating in the environment external to the earcups and removing from the recorded sounds a part of noise using said sounds propagating in the environment external to the earcups.

In one embodiment, the sounds propagating in the environment external to the earcups are previously recorded using an external sound recording element positioned on the outside of at least one of the earcups of the headphones. The recorded sounds and the sounds propagating in the external environment have been acquired simultaneously. These noise reduction embodiments advantageously allow to improve the accuracy of the physiological signals of a subject provided by the method.

When this denoise steps are implemented denoised sounds are obtained and are used in the following steps of the method for extracting the cardiac peaks and extracting the first estimation of the breathing signal from the inter-beat intervals presenting respiratory sinus arrhythmia.

According to one embodiment the method for monitoring physiological signals of a subject from sounds produced by the subject, said method comprising the following steps:
receiving recorded sounds comprising sounds originating from the chest of the subject and being transmitted by biological tissues of the subject to the ears of the subject, wherein said recorded sounds are recorded by at least one sound recording element positioned inside at least one earcup of headphones worn by the subject;
receiving signals from an accelerometer and a gyroscope being recorded simultaneously with the recorded sounds;
removing noise originating from sounds propagating in the environment external to the earcups from the recorded sounds to obtain denoised sounds;
extracting from the denoised sounds cardiac peaks, corresponding to systolic and diastolic sounds, and residual sounds comprising information generated by the breathing signal;
detecting heart beats from the cardiac peaks sounds and calculating inter-beat intervals from the heart beats;
extracting a first estimation of the breathing signal from the inter-beat intervals presenting respiratory sinus arrhythmia;
extracting a second estimation of the breathing signal from residual sounds;
extracting a third estimation of the breathing signal and motion artifacts from the signals of the accelerometer and the gyroscope;
calculating the breathing signal by combining the first, the second and the third estimation of the breathing signal.

According to one embodiment, the extraction of the cardiac peaks sounds comprises a step of enhancing the peaks in the recorded sounds and a step of detecting the cardiac peaks sounds using a discrete wavelet transform.

According to one embodiment, the step of extracting the first estimation of the breathing signal comprises the application of Fast Fourier Transform to the resampled inter-beat intervals and the selection of the low frequency component.

According to one embodiment, the step of extracting the second estimation of the breathing signal from the residual sound is performed using time-frequency analysis and periodicity detection.

According to one embodiment, the step of extracting the third estimation of the breathing signal comprises the use of principal component analysis decomposition, fast Fourier spectral computation and component detection of the signals of the accelerometer and the gyroscope.

According to one embodiment, wherein a fusion algorithm is used to combine the first, the second and the third estimation of the breathing signal.

According to one embodiment, the method further comprises the step of receiving electroencephalographic signals of the subject recorded simultaneously to the recorded sounds.

The present invention further relates to a computer program product for monitoring physiological signals of a subject, the computer program product comprising instructions which, when the program is executed by a computer, cause the computer to carry out the steps of the method according to any one of embodiments here above.

Yet the present invention relates to a computer-readable storage medium comprising instructions which, when the program is executed by a computer, cause the computer to carry out the steps of the method according to any one of embodiments here above.

In what follows, the modules are to be understood as functional entities rather than material, physically distinct, components. They can consequently be embodied either as grouped together in a same tangible and concrete component, or distributed into several such components. Also, each of those modules is possibly itself shared between at least two physical components. In addition, the modules are implemented in hardware, software, firmware, or any mixed form thereof as well.

The present invention also relates to a system for the monitoring of physiological signals of a subject from recorded sounds comprising:
an input module configured to receive:
   recorded sounds acquired using at least one sound recording element positioned inside at least one earcup of headphones worn by the subject, said recorded sound originating from a chest of the subject and being transmitted by biological tissues of the subject to ears of the subject;
   signals from an accelerometer and a gyroscope, said signals which have been acquired simultaneously with the recorded sounds;
an extraction module configured to extract from the recorded sounds cardiac peaks, corresponding to systolic and diastolic sounds, and residual sounds comprising information generated by the breathing signal;
a cardiac analysis module configured to detect the heart beats from the cardiac peaks sounds and calculating inter-beat intervals from the heart beats;
a respiratory analysis module configured to extract from the recorded sounds a first estimation of the breathing signal from the inter-beat intervals, extract a second estimation of the breathing signal from residual sounds, extract a third estimation of the breathing signal and motion artifacts from the signals of the accelerometer and the gyroscope, and calculate the breathing signal combining the first, the second and the third estimation of the breathing signal, and
an outputting module configured to provide the breathing signal and inter-beat intervals for health monitoring.

According to one embodiment, the system further comprises a denoising module configured to receive sounds propagating in the environment external to the earcups and remove from the recorded sounds a part of the noise using said sounds propagating in the environment external to the earcups.

According to one embodiment, the comprises:
an input module configured to record sounds originating from the chest of the subject and being transmitted by biological tissues of the subject to the ears of the subject, wherein the recorded sounds are recorded by at least one sound recording element positioned inside at least one earcup of headphones worn by the subject; and to record, simultaneously with the recorded sounds, signals from an accelerometer and a gyroscope;
a denoising module configured to remove noise originating from sounds propagating in the environment external to the earcups from the recorded sounds to obtain denoised sounds;
an extraction module configured to extract from the denoised sounds cardiac peaks, corresponding to systolic and diastolic sounds, and residual sounds comprising information generated by the breathing signal;
a cardiac analysis module configured to detect the heart beats from the cardiac peaks sounds and calculating inter-beat intervals from the heart beats;
a respiratory analysis module configured to extract from the denoised sounds a first estimation of the breathing signal from the inter-beat intervals, extract a second estimation of the breathing signal from residual sounds, extract a third estimation of the breathing signal and motion artifacts from the signal of the accelerometer and the gyroscope, and calculate the breathing signal combining the first, the second and the third estimation of the breathing signal.

According to one embodiment, the system further comprises headphones comprising two earcups configured to amplify the sound originating from the chest of the subject and being transmitted by biological tissues of the subject to the ears of the subject.

According to one embodiment, the at least one sound recording element is positioned inside at least one of the earcups.

According to one embodiment, the system further comprises an external sound recording element positioned on the outside of at least one of the earcups so as to record the sounds propagating in the environment external to the earcups.

According to one embodiment, the earcups of the headphones are circumaural headphones or supra-aural headphones.

According to one embodiment, the denoising module is further configured to perform noise cancellation using an adaptative filter to remove the recorded sounds propagating in the environment external to the earcups from the recorded sounds

According to one embodiment, the system comprises at least two electrodes configured to acquire the electroencephalographic signal of the subject. In one embodiment, the at least two electrodes are textile electrodes comprised in the earcups so that the textile electrodes rest against the skin disposed over the mastoid processes when the headphones is worn by a subject. the use of textile electrodes ensure comfort to the subject.

### Definitions

In the present invention, the following terms have the following meanings:
“Subject” refers to a mammal, preferably a human. In the sense of the present invention, a subject may be an individual having any mental or physical disorder requiring regular or frequent medication or may be a patient, i.e. a person receiving medical attention, undergoing or having underwent a medical treatment, or monitored for the development of a disease.
“Headphones” refer to a pair of small loudspeaker drivers worn on or around the head over a user's ears.
“Electroencephalogram” refers to the record of the electrical activity of the brain of a subject.
“Processor” this term is herein not restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.
“Real time”: refers to the ability of a system of controlling an environment by receiving data, processing them, and returning the results sufficiently quickly to affect the environment at that time. Real-time responses (i.e. output) are often understood to be in the order of milliseconds, and sometimes microseconds.

### BRIEF DESCRIPTION OF THE DRAWINGS

FIG. 1 is a work flow providing a schematic representation of the steps of the method according to one embodiment of the present invention.

FIG. 2 is a work flow providing a schematic representation of the steps performed for the extraction from the denoised sounds cardiac peaks and the residual sounds.

FIG. 3 is a work flow providing a schematic representation of the steps performed for the extraction of a first estimation of the breathing signal from inter-beat intervals.

FIG. 4 is a work flow providing a schematic representation of the steps performed for the extraction of a second estimation of the breathing signal from residual sounds.

FIG. 5 is a work flow providing a schematic representation of the steps performed for the extraction of a third estimation of the breathing signal and motion artifacts from the signals of the accelerometer and the gyroscope.

FIG. 6 is a work flow providing a schematic representation of the steps performed for calculation of the breathing signal by combining the first, the second and the third estimation of the breathing signal.

FIG. 7 is a schematic representation of the system for the monitoring of physiological signals of a subject from recorded sounds according to one embodiment of the present invention.

FIG. 8 is a schematic representation of the system for the monitoring of physiological signals of a subject from recorded sounds comprising the headphones.

### DETAILED DESCRIPTION

The following detailed description will be better understood when read in conjunction with the drawings. For the purpose of illustrating, the system is shown in the preferred embodiments and the block diagrams, comprising the steps of the method, are shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

The present invention relates to a method 100 for monitoring physiological signals of a subject from the acoustic analysis of sounds produced by physiological process in a subject. Among the multiple physiological process taking place in the human body, heart beating and respiration are among those that produce the most perceptible sounds.

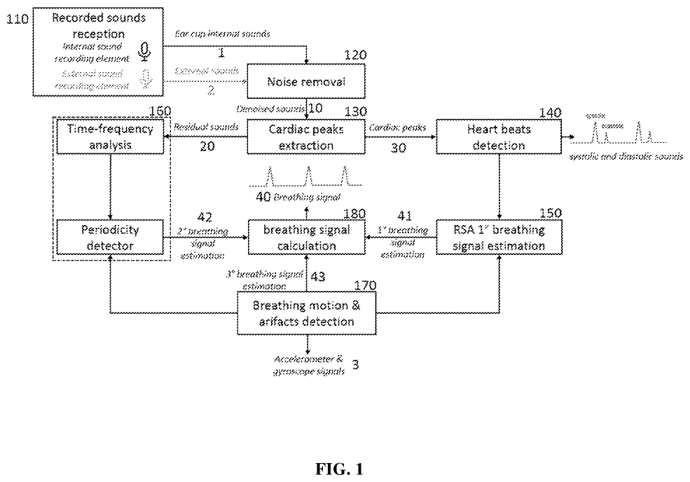

According to the embodiment illustrated in FIG. 1, an initial step of the method consists in the reception 110 of recorded sounds 1 comprising sounds originating from the chest of the subject and being transmitted by biological tissues of the subject to the ears of the subject. Said sounds originating from the chest are produced at least in part from the heart beating and respiration.

In one embodiment, the recorded sounds 1 are recorded by at least one sound recording element positioned inside at least one earcup of headphones worn by the subject, the sound recording element being notably a microphone. The earcup of the headphone generally present a curved body where the convex surface faces the ear of the subject sealing at least partially the volume between the ear and the earcup from the external environment while the concave surface faces the external environment. In one embodiment, the at least one sound recording element is disposed on the convex surface of the earcup. However, even if the shape of the earcups is configured to seal the concave surface of the earcup and the sound recording element from the external environment, the acoustic isolation is not complete, and therefore the sound recorded from the sound recording element comprises also a component originating from the sounds propagating in the environment external to the earcups 2.

According to one embodiment, the initial step 110 of the method further comprises the reception of sounds propagating in the environment external to the earcups 2. Said sounds propagating in the environment external to the earcups 2 may be recorded by using an external sound recording element positioned on the outside of at least one of the earcups of the headphones.

In one embodiment, the earcups of the headphones are circumaural headphones or supra-aural headphones. In circumaural headphones have circular or ellipsoid earcup that encompass the ears. Because these headphones completely surround the ear, circumaural headphones can be designed to fully seal against the head to attenuate external noise. Supra-aural headphones have pads that press against the ears, rather than around them. This type of headphone generally tends to be smaller and lighter than circumaural headphones, resulting in less attenuation of outside noise.

In one embodiment, the method further receives as input signals from an accelerometer and a gyroscope being recorded simultaneously with the recorded sounds. The signals may be recorded directly from an accelerometer and a gyroscope integrated into the headphones.

According to one embodiment, the method comprises a step of removal of noise 120 originating from sounds propagating in the environment external to the earcups from the recorded sounds 1 to obtain denoised sounds 10. Standard noise cancellation technique, known by the person skilled in the art.

According to the embodiment wherein the method comprises the reception of sounds propagating in the environment external to the earcups, the step of removal of noise uses an active noise cancellation algorithm using for example adaptative filtering and the sounds propagating in the environment external to the earcups.

In one embodiment, the method comprises a step of extracting 130 from the denoised sounds 10 cardiac peaks sounds 30, corresponding to systolic and diastolic sounds, and residual sounds 20 comprising information generated by the breathing signal.

According to one embodiment, the extraction of the cardiac peaks sounds comprises a step of enhancing the peaks in the recorded sounds 1 and a step of detecting the cardiac peaks sounds using a discrete wavelet transform.

Cardiac sounds are noticeable as periodic double peak, corresponding to systolic and diastolic sounds. In one embodiment, the discrete wavelet transform (DWT) is used in this step to enhance and detect the period cardiac peaks sounds 30. Because discrete wavelet transform localizes patterns in signals to different scales, relevant signal features can be preserved while removing noise. In one embodiment, a wavelet denoising based on 4^{th} order symlets is used. This pattern is matched into the original sound and extracted to obtain two signals: one related to cardiac sounds and another containing noise and respiratory sounds.

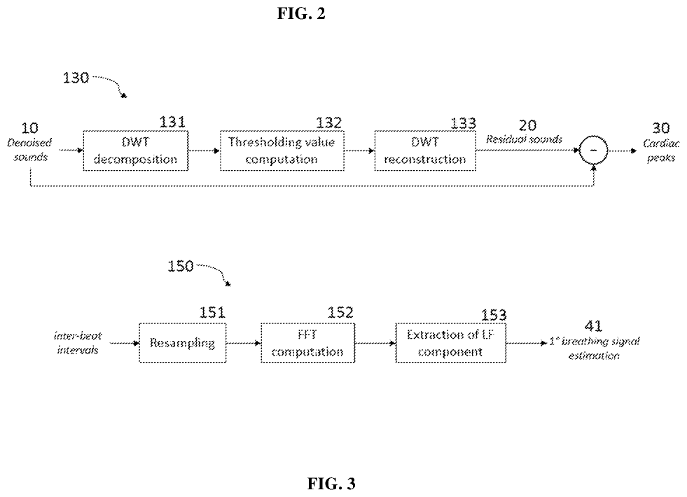

In one example illustrated in FIG. 2, the denoised sounds 10 is decomposed 131 using the wavelet of 4^{th} order symlets. Following the decomposition of the denoised sounds 10, a thresholding value is computed 132 in order to isolate the cardiac peaks sounds from other components in the sounds using a method know by the person skilled in the art such as Birgé-Massart strategy. The following step consists in the reconstruction by discrete wavelet transform 133 of the residual sounds 20, meaning sounds not comprising cardiac peaks. Finally, the cardiac peaks sounds 30 are obtained by subtraction of the residual sounds 20 from the denoised sounds 10.

According to one embodiment, the method comprises the step of detecting heart beats 140 from the cardiac peaks sounds 30.

In one example, the step 140 comprises the application of a band-pass filter to the cardiac peaks sounds 30 and the application of a derivative operation to the filtered sounds. In this example, the derivative operation is followed by a squared operation and a moving window integration of the resulting sounds. Finally, applying an adaptative threshold to the heart beats are detected.

According to one embodiment, the method further comprises the calculation of the inter-beat intervals from the heart beats. In one alternative embodiment, the inter-beat intervals are calculated from the cardiac peak sounds 30.

According to one embodiment, the method of the present invention comprises a step 150 of extracting a first estimation of the breathing signal 41 from the inter-beat intervals presenting respiratory sinus arrhythmia (RSA). Indeed, respiratory sinus arrhythmia (RSA) is a prominent component of heart rate variability, this is the phenomenon by which the R-R interval on a cardiac signal is shortened during inspiration and prolonged during expiration.

According to one embodiment illustrated in FIG. 3, the method further comprises the step of resampling the inter-beat intervals 151 and computing a fast Fourier transform of the resampled inter-beat intervals 152. In one embodiment, the first estimation of the breathing signal 41 is extracted from the selection of the low frequency components of the fast Fourier transform 153.

According to an alternative embodiment, the QRS complexes are detected using a waveform decomposition strategy. An exemplary decomposition algorithm may use a 2-poles 2-zeros resonator filter centered at 17 Hz with a bandwidth of 6 Hz to highlight the sharp edges of the QRS and smooth out the other ECG waveforms. Once filtered, a simple adaptive threshold filter may be used to select the QRS fiducial point used by the second stage of the respiration detection algorithm. After the QRS annotation, the heart rate variability may be calculated over the duration of the sampled ECG data, allocating an x-value equal to the midpoint between the R peaks. This heart rate variability is then inverted and resampled at 1000 Hz. The heart rate variability is considered to be the respiration waveform, where the local minima are considered the point of maximum expiration due to the decreased heart rate, and the local maxima are the points of maximum inspiration for each breath.

According to one embodiment, the method comprises a step 160 of extracting a second estimation of the breathing signal 42 from residual sounds 20.

According to one embodiment, the step 160 of extracting the second estimation of the breathing signal 42 from the residual sound is performed using time-frequency analysis and periodicity detection.

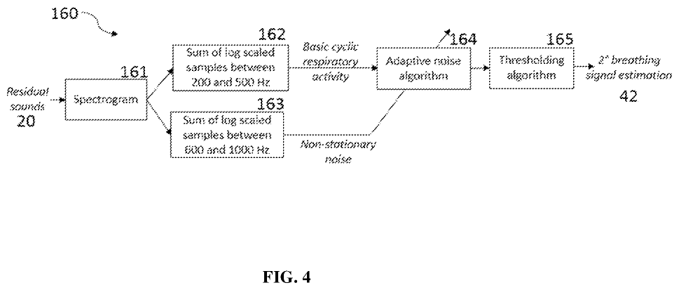

In one example illustrate in FIG. 4, the step 160 comprises a first step of obtaining a spectrogram 161 of the residual sounds 20. Two frequency regions are selected on the spectrogram: one region corresponding to the basic cyclic respiratory activity (200-500 Hz) and one region corresponding to non-stationary noise manifestations with broadband bursts in the context of the total observation time (600-1000 Hz). In this example, the integral power synthesis signal is performed in the selected frequency range 200-500 Hz as sum of log scaled samples of power spectral density 162 obtaining a synthesized signal of basic cyclic respiratory activity. For the region comprising the frequencies 600-1000 Hz, the integral power synthesis signal is performed in the selected frequency range as sum of log scaled samples of power spectral density 163 obtaining a synthesized signal of non-stationary noise. Synthesized signal of basic cyclic respiratory activity is used as reference for detection of respiratory cycles. However, the signal component may contain noise spikes, which considerably complicates detection of respiratory cycles even after the first denoising step. Indeed, in most cases, recording of respiratory sounds is associated with the imposition of secondary and non-informative components as physiological (secondary noise, wheezing) and non-physiological nature interference. Assuming that the synthesized signal of basic cyclic respiratory activity is some kind additive mixture of the actual main component of respiratory activity and component of the non-stationary noise, an adaptive noise filtering algorithm 164 using the basic cyclic respiratory activity on the second frequency band is used for the cancelation of noise. Through this approach the respiratory activity signal was obtained as filtering result, given that the desired signal respiratory activity and respiratory noise signal power bursts are not correlated with each other. In this example, the output signal of the adaptive filter is additional filtered with a cutoff frequency around 4 Hz, which eliminates the influence of high frequency components of the breathing signal. A threshold algorithm 165 is further used for the extraction of the second estimation of the breathing signal 42.

According to one embodiment, the method of the present invention comprises a step 170 of extracting a third estimation of the breathing signal 43 and motion artifacts from the signals of the accelerometer and the gyroscope 3.

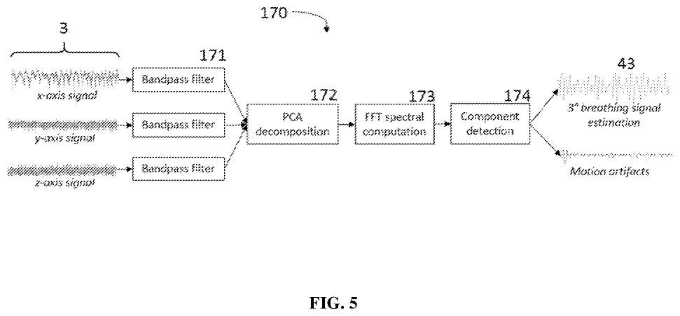

According to one embodiment illustrated in FIG. 5, the step 170 comprises the application of a bandpass filter to the signals of the accelerometer and/or the gyroscope 3, the bandpass filter 171 being applied separately to the signal recorded from the 3 axis of the accelerometer and/or the 3 axis of the gyroscope. In one embodiment, principal component analysis decomposition 172 is used with the filtered signals of the accelerometer and/or the gyroscope and for the resulting signals the fast Fourier transform spectral 173 is computed obtaining signals in the frequency domain. In one embodiment, the third estimation of the breathing signal 43 and motion artifacts are extracted from the signals in the frequency domain using component detection 174.

According to one embodiment, the method comprises a step of calculating 180 the breathing signal 40 by combining the first 41, the second 42 and the third estimation of the breathing signal 43.

According to one embodiment, a fusion algorithm is used to combine the first 41, the second 42 and the third estimation 43 of the breathing signal. The combination of estimations can be done by several ways known by the man skilled in the art, such as using an ensemble learning, in particular cascading, which concatenates individual classifiers to obtain a more accurate result.

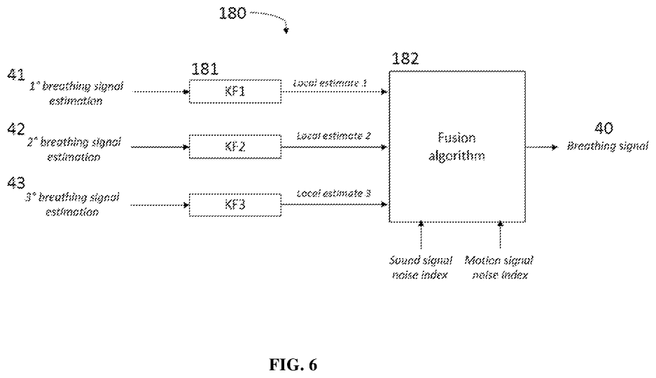

According to one example illustrated in FIG. 6, the fusion algorithm is configured to adapt to different acquisition scenarios: depending on environmental noise and movement patterns, one of the three breathing signal estimation is considered more effective and therefore its weight increased with respect the others. For each breathing signal estimation (41, 42, 43) Kalman filtering (KF) 181 is used to obtain respective local estimates. Thus, two signal quality indices evaluating the quality of the signals are computed, notably a sound signal noise index using the external sounds 2 and/or the residual sounds 20 and a motion signal noise index using the accelerometer and/or gyroscope signals 3. The two signal quality indices are used to weight each local estimate based on previous reference data (i.e. learning dataset). Local estimates from individual Kalman-filters need to be fused in a manner that takes into account the uncertainty associated with each estimate (signal quality indices) and previous information from a learning dataset. State vector fusion methods use a bank of Kalman filters to obtain local estimates which are then fused to obtain an improved breathing signal 40 which is an accurate robust estimation of the breathing signal using signal quality indices and a modified Kalman Filter fusion framework 182 which uses the signal quality indices to adaptively update the Kalman Filter noise covariance estimate. The signal quality indices are derived in real time and therefore no assumptions concerning the signal-to-noise ratio are required. The main advantage of this approach is the inclusion of a signal quality metric (as well as the past behavior of each signal) to control the Kalman Filter noise covariance estimate and decide automatically how to weight each source of information.

According to one embodiment, the method further comprises providing as output the breathing signal 40 and inter-beat intervals for monitoring the health status of the subject.

According to one embodiment, the method further comprises the step of receiving electroencephalographic signals of the subject recorded simultaneously to the recorded sounds from which are estimated the physiological signals.

According to one embodiment, electroencephalographic signals is recorded synchronized with ECG and respiration so time-locked analysis can be performed in real time. In this embodiment, it is possible for instance to correlate event related potentials using heartbeats or inspiratory marks.

The present invention further relates to a computer program product for monitoring physiological signals of a subject, the computer program product comprising instructions which, when the program is executed by a computer, cause the computer to carry out the steps of the method according to any one of the embodiments described hereabove.

The computer program product to perform the method as described above may be written as computer programs, code segments, instructions or any combination thereof, for individually or collectively instructing or configuring the processor or computer to operate as a machine or special-purpose computer to perform the operations performed by hardware components. In one example, the computer program product includes machine code that is directly executed by a processor or a computer, such as machine code produced by a compiler. In another example, the computer program product includes higher-level code that is executed by a processor or a computer using an interpreter. Programmers of ordinary skill in the art can readily write the instructions or software based on the block diagrams and the flow charts illustrated in the drawings and the corresponding descriptions in the specification, which disclose algorithms for performing the operations of the method as described above.

The present invention further relates to a non-transitory computer-readable storage medium comprising instructions which, when the computer program is executed by a data processing system, cause the data processing system to carry out the steps of the method according to any one of the embodiments described hereabove.

Computer programs implementing the method of the present embodiments can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

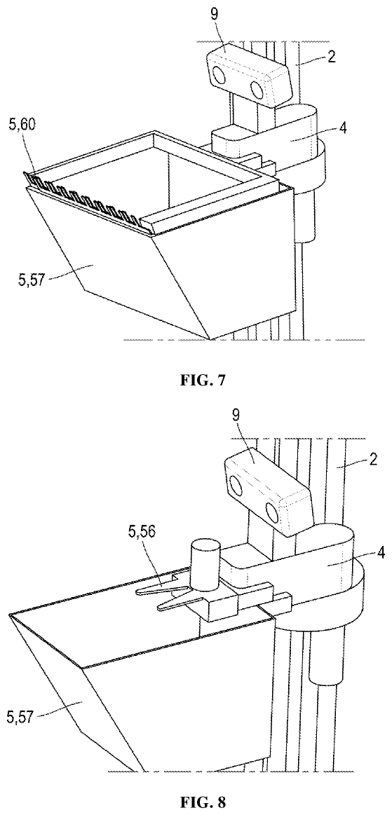

Yet another aspect of the present invention concerns a system for the monitoring of physiological signals of a subject from recorded sounds. Said system S comprises a plurality of modules cooperating with each other's as shown in FIG. 7.

According to one embodiment, the system comprises an input module AM configured to receive recorded sounds originating from the chest of the subject and being transmitted by biological tissues of the subject to the ears of the subject. Said recorded sounds 1 are recorded by means of at least one sound recording element positioned inside at least one earcup of headphones worn by the subject. In this embodiment, the input module AM is further configured to receive, simultaneously with the recorded sounds, signals recorded from an accelerometer and a gyroscope 3.

Alternatively, the input module AM is configured to record sounds originating from the chest of the subject and being transmitted by biological tissues of the subject to the ears of the subject using at least one sound recording element positioned inside at least one earcup of headphones worn by the subject. The input module AM may be further configured to record, simultaneously with the recorded sounds, signals from an accelerometer and a gyroscope 3.

As shown in FIG. 8, according to one embodiment, the system comprises headphones H comprising two earcups E. According to one embodiment, the earcups E of the headphones H are circumaural headphones or supra-aural headphones.

According to one embodiment, the at least one sound recording element RE is positioned inside one of the two earcups E of the headphones H.

In one embodiment, the earcups E are configured to amplify the sound originating from the chest of the subject and being transmitted by biological tissues of the subject to the ears of the subject. In this embodiment, the earcups E have a curved body where the convex surface faces the ear of the subject sealing at least partially the volume between the ear and the earcup from the external environment while the concave surface faces the external environment. The convex surface facing the ear of the subject is configured to be a resonant cavity capable of attenuating some frequencies, such as the frequencies associated to of the noise in the external environment, and/or to amplify some other frequencies, such as the frequencies associated to the breathing sound produced by the breathing activity of the subject. As consequence, the sounds recorded from the sound recording element RE inside one earcup is not equivalent in frequency components modulation to the sounds that are recorded from a recording element positioned on the exterior surface of an ear plug and placed inside an intra-auricular canal. The frequency components modulation obtained thanks to the use of the recording element RE inside the earcup advantageously allows to obtain a better estimation of the physiological signals.

In one advantageous embodiment, the sound recording element RE is a loud speaker having a membrane of larger dimension ranging from 1.50 to 2.5 cm, preferably 1.8 to 2.2 cm. The larger dimension of the membrane herein implemented, compared to the ones used for intra-auricular devices, allows to obtain a higher sensitivity to the sounds originating from the chest of the subject. At the same time, the sound recording element RE is external to the auditory canal, ensuring a better comfort for the subject and improved stability during utilization of the system.

According to one embodiment, the system comprises a denoising module DM configured to remove noise originating from sounds propagating in the environment external to the earcups from the recorded sounds 1 to obtain denoised sounds 10. Standard noise cancellation technique, known by the person skilled in the art, may be implemented in this module.

According to one embodiment, the system further comprises an external sound recording element RE positioned on the outside of at least one of the earcups so as to record the sounds propagating in the environment external to the earcups 2. According to this embodiment, the denoising module DM is configured to implement an active noise cancellation algorithm using for example adaptative filtering and the sounds propagating in the environment external to the earcups 2.

According to one embodiment, the system comprises an extraction module EM configured to extract from the denoised sounds 10 cardiac peaks 30, corresponding to systolic and diastolic sounds, and residual sounds 20 comprising information generated by the breathing signal.

According to one embodiment, the extraction module EM is configured to enhance the peaks in the recorded sounds 1 and to detect the cardiac peaks sounds using a discrete wavelet transform. In one embodiment, the extraction module EM is configured to use the discrete wavelet transform (DWT) to enhance and detect the period cardiac peaks sounds 30. Because discrete wavelet transform localizes patterns in signals to different scales, relevant signal features can be preserved while removing noise. In one embodiment, a wavelet denoising based on 4^{th} order symlets is used. This pattern is matched into the original sound and extracted to obtain two signals: one related to cardiac sounds and another containing noise and respiratory sounds.

In one example, the extraction module EM is configured to decompose the denoised sounds 10 using the wavelet of 4^{th} order symlets, compute a thresholding value in order to isolate the cardiac peaks sounds from others components in the sounds using known method, such as Birgé-Massart strategy, and to reconstruct by discrete wavelet transform the residual sounds 20, being sounds not comprising cardiac peaks.

Finally, the extraction module EM is configured to obtain the cardiac peaks sounds 30 are by the subtraction of the residual sounds 20 from the denoised sounds 10.

According to one embodiment, the system comprises a cardiac analysis module CAM configured to detect the heart beats from the cardiac peaks sounds 30 and calculating inter-beat intervals from the heart beats.

In one embodiment, the cardiac analysis module CAM is configured to band-pass filter the cardiac peaks sounds 30 and to implement the following operation on the filtered signals to detect the heart beats: a derivative operation, a squared operation, a moving window integration and an adaptative threshold. The cardiac analysis module CAM is further configured to calculation of the inter-beat intervals from the heart beats.

According to one embodiment, the system further comprises a respiratory analysis module RAM configured to extract from the denoised sounds 10 a first estimation of the breathing signal from the inter-beat intervals, to extract a second estimation of the breathing signal from residual sounds 20, to extract a third estimation of the breathing signal and motion artifacts from the signal of the accelerometer and the gyroscope, and to calculate the breathing signal combining the first, the second and the third estimation of the breathing signal

According to one embodiment, the respiratory analysis module RAM is configured to resample the inter-beat intervals, compute a fast Fourier transform of the resampled inter-beat intervals and extract the first estimation of the breathing signal from the low frequency components of the fast Fourier transform.

According to one embodiment, the respiratory analysis module RAM is configured to extract the second estimation of the breathing signal from the residual sound 20 using time-frequency analysis and periodicity detection.

According to one example, the respiratory analysis module RAM is configured to perform a first step to obtain a spectrogram of the residual sounds 20. Two frequency regions are selected on the spectrogram: one region corresponding to the basic cyclic respiratory activity (200-500 Hz) and one region corresponding to non-stationary noise manifestations with broadband bursts in the context of the total observation time (600-1000 Hz). In this example, the respiratory analysis module RAM further performs the integral power synthesis signal in the selected frequency range 200-500 Hz as sum of log scaled samples of power spectral density obtaining a synthesized signal of basic cyclic respiratory activity. For the region comprising the frequencies 600-1000 Hz, the integral power synthesis signal is performed by the respiratory analysis module RAM in the selected frequency range as sum of log scaled samples of power spectral density obtaining a synthesized signal of non-stationary noise. The synthesized signal of basic cyclic respiratory activity is used as reference for detection of respiratory cycles. An adaptive noise filtering algorithm using the basic cyclic respiratory activity on the second frequency band is used for the cancelation of noise by the respiratory analysis module RAM. This approach allows to obtain the respiratory activity signal as filtering result, given that the desired signal respiratory activity and respiratory noise signal power bursts are not correlated with each other. In this example, the respiratory analysis module RAM is further configured to filtered with a cutoff frequency around 4 Hz the output signal of the adaptive filter in order to eliminate the influence of high frequency components of the breathing signal. A threshold algorithm is further used for the extraction of the second estimation of the breathing signal 42.

According to one embodiment, the respiratory analysis module RAM is configured to extract a third estimation of the breathing signal and motion artifacts from the signal of the accelerometer and the gyroscope 3.

In one embodiment, the respiratory analysis module RAM is configured to bandpass filter separately the signals of the accelerometer and/or the gyroscope 3 recorded from the three axis of the accelerometer and/or the three axis of the gyroscope. In one embodiment, the respiratory analysis module RAM is configured to perform principal component analysis decomposition with the filtered signals of the accelerometer and/or the gyroscope and compute fast Fourier spectral transform to obtain signals in the frequency domain According to one embodiment, the third estimation of the breathing signal and motion artifacts are extracted by the respiratory analysis module RAM from the signals in the frequency domain using component detection.

According to one embodiment, the method comprises a step of calculating the breathing signal by combining the first, the second and the third estimation of the breathing signal.

According to one embodiment, the respiratory analysis module RAM is configured to use a fusion of classifications to combine the first, the second and the third estimation of the breathing signal.

According to one embodiment, the system further comprises an outputting module configured to provide the breathing signal and inter-beat intervals for health monitoring.

While various embodiments have been described and illustrated, the detailed description is not to be construed as being limited hereto. Various modifications can be made to the embodiments by those skilled in the art without departing from the true spirit and scope of the disclosure as defined by the claims.

1.-17. (canceled)

18. A computer-implemented method for providing an estimation of physiological signals of a subject, said method comprising:
receiving recorded sounds comprising sounds originating from a chest of a subject and being transmitted by biological tissues of the subject to the ears of the subject, wherein said recorded sounds are previously recorded by at least one sound recording element positioned inside at least one earcup of headphones worn by the subject;
receiving signals from an accelerometer and a gyroscope which have been recorded simultaneously with the recorded sounds;
extracting from the recorded sounds cardiac peaks, corresponding to systolic and diastolic sounds, and residual sounds comprising information generated by respiration of the subject;
detecting heart beats from the cardiac peaks sounds and calculating inter-beat intervals from the heart beats;
extracting a first estimation of a breathing signal from the inter-beat intervals presenting respiratory sinus arrhythmia;
extracting a second estimation of the breathing signal from residual sounds;
extracting a third estimation of the breathing signal and motion artifacts from the signals of the accelerometer and the gyroscope;
calculating an estimation of the breathing signal by combining the first, the second and the third estimation of the breathing signal, and
providing the estimation of the breathing signal for health monitoring.

19. The method according to claim 18, wherein extracting of the cardiac peaks sounds comprises enhancing the peaks in the recorded sounds and detecting the cardiac peaks sounds using a discrete wavelet transform.

20. The method according to claim 18, wherein extracting the first estimation of the breathing signal comprises the application of Fast Fourier Transform to the resampled inter-beat intervals and the selection of the low frequency component.

21. The method according to claim 18, further comprising receiving sounds propagating in an environment external to the at least one earcup of the headphones and removing from the recorded sounds a part of a noise using said sounds propagating in the environment external to the earcups.

22. The method according to claim 18, wherein extracting the second estimation of the breathing signal from the residual sounds is performed using time-frequency analysis and periodicity detection.

23. The method according to claim 18, wherein extracting the third estimation of the breathing signal comprises the use of principal component analysis decomposition, fast Fourier spectral computation and component detection of the signals of the accelerometer and the gyroscope.

24. The method according to claim 18, wherein a fusion algorithm is used to combine the first, the second and the third estimation of the breathing signal.

25. The method according to claim 18, further comprising receiving electroencephalographic signals of the subject recorded simultaneously to the recorded sounds.

26. A non-transitory computer-readable storage medium for monitoring physiological signals of a subject, the non-transitory computer-readable storage medium comprising instructions which when executed by a computer, cause the computer to carry out the method according to claim 18.

27. A system for providing an estimation of physiological signals of a subject from recorded sounds comprising:
an input module configured to receive:
recorded sounds acquired using at least one sound recording element positioned inside at least one earcup of headphones worn by the subject, said recorded sound originating from a chest of the subject and being transmitted by biological tissues of the subject to the ears of the subject; and
signals from an accelerometer and a gyroscope, said signals which have been acquired simultaneously with the recorded sounds;
an extraction module configured to extract from the recorded sounds cardiac peaks, corresponding to systolic and diastolic sounds, and residual sounds comprising information generated by respiration of the subject;
a cardiac analysis module configured to detect the heart beats from the cardiac peaks sounds and calculating inter-beat intervals from the heart beats; and
a respiratory analysis module configured to extract from the denoised sounds a first estimation of a breathing signal from the inter-beat intervals, extract a second estimation of the breathing signal from residual sounds, extract a third estimation of the breathing signal and motion artifacts from the signals of the accelerometer and the gyroscope, and calculate an estimation of the breathing signal combining the first, the second and the third estimation of the breathing signal.

28. The system according to claim 27, further comprising headphones comprising at least one earcups configured to amplify the sound originating from the chest of the subject and being transmitted by biological tissues of the subject to the ears of the subject.

29. The system according to claim 28, wherein the at least one sound recording element is positioned inside at least one of the earcups.

30. The system according to of claim 28, further comprising an external sound recording element positioned on the outside of at least one of the earcups so as to record sounds propagating in an environment external to the at least one earcups.

31. The system according to claim 28, wherein the earcups of the headphones are circumaural headphones or supra-aural headphones.

32. The system according to claim 30, further comprising a denoising module configured to receive the sounds propagating in the environment external to the earcups and remove from the recorded sounds a part of a noise using said sounds propagating in the environment external to the earcups.

33. The system according to claim 28, wherein the respiratory analysis module is further configured to apply a Fast Fourier Transform to the resampled inter-beat intervals and to select a low frequency component for extracting said first estimation of the breathing signal.

34. The system according to claim 28, wherein the respiratory analysis module is further configured to extract said second estimation of the breathing signal from the residual sounds by using time-frequency analysis and periodicity detection.

35. The system according to claim 28, wherein the respiratory analysis module is further configured to extract said third estimation of the breathing signal by using principal component analysis decomposition, fast Fourier spectral computation and component detection of the signals of the accelerometer and the gyroscope.

36. The system according to claim 28, wherein the respiratory analysis module is further configured to combine said first, said second and said third estimation of the breathing signal by using a fusion algorithm.
